(19) United States
(12) Patent Application Publication
(10) Pub. No.: US 2004/0236143 A1
(43) Pub. Date: Nov. 25, 2004

(54) Method for producing (meth)acrylic acid esters

(76) Inventors: Hans Martan, Frankenthal (DE); Gerhard Nestler, Wien (AT)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/486,364

(22) PCT Filed: Sep. 3, 2002
(86) PCT No.: PCT/EP02/09814

(30) Foreign Application Priority Data
Sep. 10, 2001 (DE) 101 44 490.7

(51) Int. Cl. C07C 69/52; C07C 67/48

(52) U.S. Cl. 560/205; 560/218

(57) ABSTRACT

(Meth)acrylates are prepared by reacting (meth)acrylic acid with an alcohol in the presence of at least one acidic catalyst and at least one polymerization inhibitor in a reaction zone b) consisting of at least one reactor b1) having at least one connected distillation unit b2), by a process in which

in reactor b1), the temperature is brought to from 120 to 150° C. and

the desired (meth)acrylic acid: alcohol stoichiometry is established,

the distillate of the distillation unit b2) is treated with a wash liquid (wash step c)),

the discharge from the wash step c) is separated into at least one organic phase c1) and at least two aqueous phases c2) and c3),

the organic phase c1) is passed partly as reflux into the distillation unit b2) and partly, without further scrubbing, into a purification b) of the (meth)acrylate by distillation and

the aqueous phases c2) and c3) are passed at least partly into an alcohol recovery d).

The present invention relates to a process for the preparation of (meth)acrylates by reacting alcohols with a substantially anhydrous crude (meth)acrylic acid in the presence of at least one strongly acidic catalyst and of an inhibitor or inhibitor mixture in the homogeneous, liquid phase, in which highly pure (meth)acrylates can be obtained in high yield in spite of the use of crude (meth)acrylic acid.

Crude (meth)acrylic acid is understood here as meaning the (meth)acrylic acid-containing mixture which occurs after absorption of the reaction gases of the propane/propene/acrolein or isobutane/isobutene/methacrolein oxidation in an absorbent and subsequent separating off of the absorbent or which is obtained by fractional condensation of the reaction gases.

Even with the use of crude (meth)acrylic acid, the resulting (meth)acrylates are obtained in high purity and substantially free of ethers, saturated esters, e.g. acetates and propionates, aldehydic and other carbonyl-containing secondary components and high-boiling secondary components.

The polymers or copolymers prepared on the basis of (meth)acrylates are of considerable commercial importance, generally in the form of polymer dispersions, for example as adhesives, surface coatings or textile, leather and paper assistants. For applications, in particular in the food or cosmetics sector, the polymer dispersions should be substantially free of volatile impurities, such as lower aldehydes, in particular C₁-C₄-aldehydes, such as formaldehyde, acetaldehyde, propionaldehyde, acrolein, methacrolein and isobutyraldehyde, or furfural, benzaldehyde and other carbonyl-containing secondary components, e.g. acetone, and acetic acid, propionic acid, protoanemonin (5-methylene-2(5H)-furanone), and ethers of the alcohol used, acetates, propionates, maleates and Michael adducts of the alcohol used and of the (meth)acrylic acid (alkoxy- and acryloyloxypropionic esters and their higher homologs), the acetates, propionates and maleates being formed by esterification of the acetic, propionic and maleic acid present in the crude (meth)acrylic acid.

For ecological and economic reasons, extremely pure (meth)acrylates are therefore required as starting materials, so that undesired impurities do not have to be removed subsequently, for example by expensive chemical or physical deodorization.

The preparation of lower (meth)acrylates by esterification of (meth)acrylic acid with lower alcohols in the presence of strong acids is generally known. The (meth)acrylic acid used is as a rule pure or prepurified (meth)acrylic acid, cf. for example Chem Systems, Acrylic Acid/Acrylates 96/97-8, November 1997, page 24.

Pure or prepurified (meth)acrylic acid is generally understood as meaning (meth)acrylic acid whose purity is at least 99.5% by weight and which is substantially free of aldehydic, other carbonyl-containing and high-boiling components. The purification of this (meth)acrylic acid is effected as a rule by multistage crystallization or, if required, by chemical treatment with an aldehyde scavenger and distillation.

Since the formation of the ester from (meth)acrylic acid and alcohol is known to be based on an equilibrium reaction, as a rule one feedstock is used in excess and/or the resulting water of esterification and/or the desired ester are removed from the equilibrium in order to obtain economical conversions. The influencing of the esterification equilibrium by the use of an excess of alcohol is however disadvantageous since, inter alia, this promotes the formation of ethers from the starting alcohols and of Michael adducts (cf. for example U.S. Pat. No. 4,280,010, column 1).

Since (meth)acrylic compounds readily tend to undergo undesired polymerization, particularly under the action of heat, in general considerable efforts are being made to avoid the formation of polymer during the esterification and isolation of the desired ester.

As a rule, this polymer formation does in fact lead to coating of the reactor walls, heat exchanger surfaces and column trays (fouling) and to blockage of pipes, pumps, valves, etc. (EP-A 522 709, page 2, lines 9-18; U.S. Pat. No. 5,171,888, column 1, lines 19-38). This results in expensive shutdowns and complicated cleaning operations, for example boiling with basic solutions which then have to be disposed of in an expensive manner, as described in DE-A 195 36 179.

In the Encyclopedia of Chemical Technology, Kirk-Othmer, 4th Ed., 1994, Vol. 1, pages 301-302, W. Bauer jr. describes a process in which the esterification is carried out in the presence of sulfuric acid at 80° C., the desired ester, the water of reaction and the excess alcohol are separated off by distillation, a part of the distillate is recycled as reflux into the column attached to the reactor and the remainder is fed to a scrubbing column, the scrubbed moist ester phase is dewatered in a distillation column with a large reflux and the desired ester is isolated from the anhydrous bottom product in a further distillation step. The bottom product obtained again is separated into a desired product fraction, which is recycled into the ester distillation, and a bottom fraction, which is discharged. A stream is taken up continuously from the reactor and is freed from the high boilers in a distillation step, the distillate being recycled into the esterification. All aqueous phases obtained are combined and the alkanol dissolved therein is recovered by distillation.

This process requires at least a prepurified acrylic acid, since otherwise the concentrations of readily volatile acetates and propionates increase as a result of reflux in the column attached to the reactor.

In Ullmann's Encyclopedia of Industrial Chemistry, 5th Ed., 1985, Vol. A1, pages 167-168, it is pointed out that, in the preparation of lower esters (methyl and ethyl acrylate), cation exchange resins are more advantageous as catalysts than sulfuric acid, which in turn is preferred in the preparation of higher esters. The process described comprises carrying out the esterification in a fixed cation exchanger bed at 60-80° C. and working up the liquid reactor discharge by distillation, comparable with the process described above.

U.S. Pat. No. 5,187,309 proposes an esterification process which starts from aqueous acrylic acid (30-50% of water). The esterification is effected at 50-110° C. in the presence of an acid as a catalyst, and the esterification mixture is fed together with the aqueous acrylic acid to a distillation unit. Acrylic acid is obtained as a bottom product and water, alkanol and acrylate are condensed at the top of the column and then further worked up. The acrylic acid is recycled directly into the esterification or a part-stream is purified beforehand by distillation (removal of high boilers and polymer). The ester-containing mixture is washed alkanol-free with water and the desired ester is then isolated therefrom by distillation.

The disadvantage of the process is that large losses of desired product (about 5.9%, based on ester production, cf. example 1, table 1, stream F9) occur, large amounts of residue result (about 4.5%, based on desired ester, stream F4) and a high energy demand is necessary since large amounts of water (water of esterification and water of the aqueous acrylic acid; about 3 mol/mol of ester) have to be evaporated. The acrylic acid losses in the washwater are also high (about 1%, based on ester, stream F7).

U.S. Pat. No. 3,875,212 proposes the use of an organic solvent having a boiling point of 100-160° C. in the esterification in order to remove the water of esterification as an azeotropic mixture from the reaction mixture. This is said to permit the use of sulfuric acid as a catalyst, to facilitate the establishing of a constant catalyst concentration and to result in a polymer which can be more readily handled. The process comprises esterifying the acrylic acid with the alkanol in the presence of sulfuric acid and of the solvent and separating off the water of esterification continuously by distillation as an azeotropic mixture with the acrylate and the solvent. The reactor discharge separates into an aqueous phase and an organic phase, which is subjected to a steam distillation in order to recover the acrylate and the solvent and to separate them from the polymer and high boilers. The aqueous phase, which contains the unconverted acrylic acid, the sulfuric acid and polymer, is filtered to separate off the polymer and is recycled to the reactor.

The process is technically complicated and inconvenient, for example owing to the necessity of a steam distillation and is therefore of no commercial importance.

U.S. Pat. No. 4,280,010 describes a process for the preparation of ether-free lower acrylates, in which the esterification is carried out at 80-130° C. in the presence of 0.1-3% of sulfuric acid or 1-8% of sulfonic acid and with an acrylic acid alkanol ratio of 1:1-2. Preferably, a reaction temperature of 85-110° C. is proposed since the ether formation and the tendency to polymerization increase at higher temperature (column 2, lines 53-57). The desired ester, the water of esterification and the alkanol are removed in gaseous form and condensed. The condensate separates into two phases, the aqueous phase being separated off and some of the organic phase being worked up by distillation and some of it being used as reflux. First, the alkanol is separated off together with the desired ester as an azeotropic mixture and is recycled into the reactor and then the desired ester is isolated from the bottom product.

The disadvantage of this process is that pure acrylic acid has to be used and that large amounts of residue are obtained (3-7%, based on desired ester).

U.S. Pat. No. 4,076,950 describes a process for the preparation of acrylates by acid-catalyzed esterification of acrylic acid with alkanols and isolation of a crude ester distillate which consists of desired ester, water of esterification and excess alkanol, in which substantially anhydrous ester is fed to the reactor in order to obtain an acrylic acid-free crude ester distillate and in order to maintain the composition of the ester/alkanol and ester/water azeotropic mixtures. The crude ester distillate must then also be subjected to a polish neutralization in which the otherwise usual phase separation problems and problems associated therewith in the working up by distillation occur only to a small extent. The crude ester distillate is washed with water during the condensation, and the organic ester phase is washed again with water after the phase separation and then dewatered by distillation.

The process requires the use of prepurified ester for maintaining the azeotropic mixtures in the reactor and needs two wash processes and a plurality of distillation stages and is therefore uneconomical. Furthermore, it does not permit the use of crude acrylic acid.

U.S. Pat. No. 4,464,229 proposes carrying out the esterification of the acrylic acid with less than the molar amount of alkanol (1:0.5-1). The process comprises separating the acrylate, water and alkanol from the esterification mixture, consisting of acrylic acid, acrylate, water and alkanol, via the top in a distillation column, anhydrous and alkanol-free acrylate being used as reflux (bottom product of the low boiler removal). The amount of reflux is established so that a specific water content is reached in the distillate. In the case of methyl acrylate, this should be 3-8%. However, this means that 116-477 kg of methyl acrylate prepurified by distillation are required as reflux per 100 kg of methyl acrylate formed.

After the water of esterification has been separated off, the organic phase of the distillate is fed to a low boiler distillation, if required after carrying out an extraction (cf. column 4, line 21). The bottom product, mainly comprising acrylic acid, is at least from 200 to 600% of the amount of freshly fed-in (meth)acrylic acid and is recycled into the esterification reactor, preferably after removal of high-boiling byproducts and polymer by distillation. The condensate of the low boiler distillation separates into an aqueous phase, which is discharged, and an organic phase, which is completely recycled as reflux. The bottom product of the low boiler column is partly used as reflux in the first distillation column (see above) and the remainder is worked up in a further distillation step to give acrylate. Aqueous phases obtained can be worked up in an alcohol recovery, which is not explained in more detail.

The process is complicated, it requires pure (meth)acrylic acid as starting material and large amounts of (meth)acrylic acid and large amounts of ester prepurified by distillation are circulated as refluxes.

It is an object of the present invention to provide a simple and economical process for the preparation of (meth)acrylates, in which it is possible to start from crude (meth)acrylic acid, which is technically simple and gives a high yield and highly pure products and in which little waste and very little polymer result.

We have found that this object is achieved by a process for the preparation of (meth)acrylates by reacting (meth)acrylic acid with an alcohol in the presence of at least one acidic catalyst and at least one polymerization inhibitor in a reaction zone b) consisting of at least one reactor b1) having at least one connected distillation unit b2), wherein

in the reactor b1), the temperature is brought to from 120 to 150° C. and

the desired (meth)acrylic acid:alcohol stoichiometry is established,

the distillate of the distillation unit b2) is treated with a wash liquid (wash step c)),

the discharge from the wash step c) is separated into at least one organic phase c1) and at least two aqueous phases c2) and c3),

some of the organic phase c1) is passed as reflux in the distillation unit b2) and some of it is passed without further washing into a purification g) of the (meth)acrylate by distillation and

the aqueous phases c2) and c3) are passed at least partly into an alcohol recovery d).

The novel process achieves the object and has the following features:

1. A crude (meth)acrylic acid can be used a starting material.

2. The esterification is carried out at a temperature at which the Michael adducts are partly cleaved back into the parent components.

3. A mixture of desired ester, water, alcohol, alkoxyester and low boilers is discharged continuously from the reactor b1) via a connected distillation unit b2).

4. Michael adducts are separated off in the alcohol recovery column d) and recycled into the reactor b1).

5. The desired ester contained in the low boiler fraction of the low boiler removal e) can be recovered in a further distillation step (low boiler working-up f) as bottom product f2) and some of it can be introduced into the low boiler removal e) and some of it can be applied as reflux to the reactor column b2).

6. The bottom product b3) discharged from the reactor contains not more than 10% of desired ester and not more than 15% of (meth)acrylic acid.

7. The residue b3) discharged can be fed to a residue working-up (stage 8) and/or to a sulfuric acid recovery.

The novel process can be used for the preparation of both methacrylates and acrylates, preferably for the preparation of acrylates, and is described below by way of example for acrylates.

The preparation of the crude (meth)acrylic acid is effected in a manner known per se, as a rule by heterogeneously catalyzed gas-phase partial oxidation of at least one C₃- or C₄-precursor of (meth)acrylic acid, e.g. propane, propene, acrolein or isobutane, isobutene or methacrolein, with molecular oxygen at elevated temperatures.

For this purpose, in the preparation of (meth)acrylic acid, the starting gas is as a rule diluted with gases inert under the chosen reaction conditions, e.g. nitrogen (N₂), CO₂, saturated C₁-C₆-hydrocarbons and/or steam, and, as a mixture with molecular oxygen (O₂) or an oxygen-containing gas, is passed at elevated temperatures (usually from 200 to 450° C.) and, if required, superatmospheric pressure over solid transition metal mixed oxide catalysts, for example those containing Mo and V or Mo, W, Bi and Fe, and converted into (meth)acrylic acid by oxidation. These reactions can be carried out in one or more stages with in each case 1, 2 or more reaction zones and/or catalyst beds which may have a composition and/or reactivity variable from reaction zone to reaction zone. Exemplary processes are described, for example, in DE-A 19 62 431, DE-A 29 43 707, DE-C 12 05 502, EP-A 257 565, EP-A 253 409, DE-A 22 51 364, EP-A 117 146, GB-B 1 450 986 and EP-A 293 224.

Methacrolein can of course also be obtained by aldol condensation of propionaldehyde and formaldehyde and then converted into methacrylic acid, for example as described above.

The acrylic acid-containing product gas mixture used according to the invention is preferably obtained from the partial oxidation of propane, propene and/or acrolein.

The corresponding hot reaction mixture contains the abovementioned lower aldehydes and water and a high proportion of noncondensable components, such as carbon oxides, nitrogen or oxygen, in addition to the (condensable) acrylic acid and condensable secondary components, e.g. acetic acid, propionic acid, acetone, acrolein and allyl acrylate.

Numerous processes are known for separating the acrylic acid from such a reaction gas mixture. Thus, for example in DE-C 21 36 396 or DE-A 24 49 780, the acrylic acid is separated from the reaction gases obtained in the catalytic gas-phase oxidation by countercurrent absorption using a high-boiling hydrophobic solvent. A crude acrylic acid is separated off by distillation from the resulting acrylic acid-containing mixture. Absorption of acrylic acid in high-boiling solvents is also described, for example, in DE-A 2 241 714 and DE-A 43 08 087.

DE-A 2 241 714 describes the use of esters of aliphatic or aromatic mono- or dicarboxylic acids which have a melting point of less than 30° C. and a boiling point at atmospheric pressure of above 160° C.

DE-A 43 08 087 recommends the use of a high-boiling mixture of from 0.1 to 25% by weight of ortho-dimethyl phthalate, based on a mixture consisting of from 70 to 75% by weight of diphenyl ether and from 25 to 30% by weight of biphenyl, for separating acrylic acid and reaction gases of the catalytic oxidation by countercurrent absorption.

Absorption of the reaction gas in water or aqueous acrylic acid solution as an absorbent is also widely used.

The crude acrylic acid is then obtained by separation from the absorbent by distillation.

After the absorption or before the distillation, the absorbed acrylic acid can also be subjected to a desorption or stripping process in order to reduce the content of aldehydic or other carbonyl-containing secondary components.

It is also possible to subject the reaction mixture from the catalytic gas-phase oxidation for the preparation of acrylic acid to fractional condensation by passing it from below into a column having internals with separation activity and condensing out the condensable components by cooling, as described, for example, in DE-A 197 40 253, or by a process analogous to this, in which the high boiler fraction is removed via a side take-off, as described in the German Application having the application number 10053086.9.

The crude acrylic acid which can be used according to the invention is preferably obtained via a fractional condensation or by absorption in diphenyl ether/biphenyl/phthalate mixtures.

The process by means of which the usable crude (meth)acrylic acid has been obtained is unimportant with regard to the novel process.

The novel process comprises the following stages:

1. Pretreatment (Optional)

The crude acrylic acid prepared by any desired process and used in the novel process or another acetic acid- or propionic acid-containing acrylic acid stream may contain, for example, the following components:

| | |
|---|---|
| Acrylic acid | 90-99.9% by |
| | weight |
| Acetic acid | 0.05-3% by |
| | weight |
| Propionic acid | 0.01-1% by |
| | weight |
| Diacrylic acid | 0.01-5% by |
| | weight |
| Water | 0.05-10% by |
| | weight |
| Furfural | 0.01-0.1% by |
| | weight |
| Benzaldehyde | 0.01-0.05% by |
| | weight |
| Other aldehydes and | 0.01-0.3% by |
| other | weight |
| carbonyl-containing | 0.01-0.1% by |
| inhibitors | weight |
| Maleic acid(anhydride) | 0.001-0.5% by |
| | weight |

When such a crude acrylic acid is used, it is advantageously treated, before use in the esterification, in the presence of an amine, hydrazine or hydrazine derivative, preferably a primary or secondary amine or hydrazine (derivative) and particularly preferably a hydrazine, in amounts of 0.5-2 mol/mol of carbonyl-containing impurities, preferably 1-2 and particularly preferably 1-1.5, mol/mol, and at 20-40° C. for 0.1-10, preferably 0.5-7, particularly preferably 1-5, hours.

Aminophenols, aminoguanidine and salts thereof, e.g. aminoguanidine bicarbonate, carboxylic acid hydrazides, e.g. adipic acid dihydrazide, aniline, monoethanolamine, diethanolamine, hydrazine, hydrazine hydrate, phenylhydrazine, 4-nitrophenylhydrazine and 2,4-dinitrophenylhydrazine are preferably used, particularly preferably hydrazine hydrate.

The reaction is preferably carried out in the presence of 300-3 000 ppm of phenothiazine as a stabilizer or of an equivalent amount of another suitable stabilizer.

In order to avoid additional apparatuses, the reaction can be carried out, for example, in a storage tank or a receiver or intermediate container, which is preferably provided with circulation, stirring or a pumped circulation.

Alternatively, the pretreatment can also be carried out in a tubular reactor which may have either trace heating, for example by means of a double jacket, or be thermally insulated after heating in the entrance region, for example by means of a heat exchanger.

If the starting acid used is an aldehyde-free acrylic acid-containing fraction obtained in the purification of crude acrylic acid in the preparation of pure acrylic acid and having a carbonyl content of less than 50 ppm, preferably less than 10 ppm, the pretreatment described can be dispensed with.

Of course, a pure acrylic acid can also be used for the novel process in which case no pretreatment is generally carried out.

Such pure acrylic acids may have, for example, the following composition:

| | |
|---|---|
| Acrylic acid | 99.7-99.9% by |
| | weight |
| Acetic acid | 50-1500 ppm by |
| | weight |
| Propionic acid | 10-500 ppm by |
| | weight |
| Diacrylic acid | 10-1000 ppm by |
| | weight |
| Water | 50-1000 ppm by |
| | weight |
| Aldehydes and other | 1-50 ppm by weight |
| carbonyl-containing | 100-300 ppm by |
| inhibitors | weight |
| Maleic acid (anhydride) | 1-20 ppm by weight |

2. Esterification

The acrylic acid-containing mixture which may originate from the pretreatment (stage 1) is reacted in the presence of at least one acidic catalyst with the alcohol in a reaction zone b).

Suitable acidic catalysts are sulfuric acid, para-toluenesulfonic acid, benzenesulfonic acid, dodecylbenzenesulfonic acid, methanesulfonic acid or mixtures thereof, acidic ion exchangers also being possible.

Sulfuric acid, para-toluenesulfonic acid and methanesulfonic acid are preferably used, particularly preferably sulfuric acid.

The catalyst concentration is, for example, from 1 to 20, preferably from 5 to 15, % by weight, based on the reaction mixture.

Alcohols suitable for the reaction are those which have 1 to 8, preferably 1 to 4, particularly preferably 1 to 3, carbon atoms.

Methanol, ethanol, n-propanol, isopropanol, n-butanol, isobutanol and 2-ethylhexanol are preferably used, methanol and ethanol being particularly preferred.

The alcohol may be fed in as a liquid and/or gas.

The esterification takes place in at least one heatable reactor b1), thorough mixing being ensured by suitable measures. If a plurality of reactors is used, for example from two to four, they may be arranged in a cascade.

The reaction preferably takes place in one reactor.

The reactor b1) is connected to at least one distillation unit, which preferably has 30-50 theoretical plates. The distillation unit b2) is preferably mounted on the reactor b1).

It is also possible for a plurality of reactors to be connected to one distillation unit. The reflux from the distillation unit is then preferably recycled into the first reactor.

The distillation unit is of a design known per se and has the conventional internals. Suitable column internals are in principle all conventional internals, for example trays, stacked packings and/or dumped packings. Among the trays, bubble trays, sieve trays, valve trays, Thormann trays and/or dual-flow trays are preferred; among the dumped packings, those comprising rings, coils, saddles, Raschig, Intos or Pall rings, barrel or Intalox saddles, Top-Pak etc. or braids are preferred.

The condenser, if present, is of a conventional design.

A preferred embodiment comprises using the bottom region and the evaporator of a distillation unit as reactor b1).

If the alcohol is fed in as a gas (see below), the preferred metering point is below the internals of the distillation unit b2) which have separation activity or in the circulation.

The reaction mixture is stabilized against undesired polymerization by means of a suitable stabilizer, e.g. phenothiazine (0.05-0.5%, based on reaction mixture), the stabilizer preferably being fed in with the acrylic acid.

The reaction takes place at 120-150° C. and ambient pressure, but higher or reduced pressure may also be used, ambient pressure being preferred.

The reaction time is as a rule 0.5-10, preferably 1-6, hours.

The starting materials acrylic acid and alcohol are metered in as a rule in the stoichiometry 1:0.7-3.0, preferably 1:0.9-2.5, particularly preferably 1:1.0-2.0, in particular 1:1.0-1.5.

The desired ester formed in the esterification, low boilers, the Michael adducts, among these preferably the alkoxypropionates, and the resulting water of reaction are separated off as top product via the column b2) connected to the esterification reactor b1) (top temperature 70-90° C., top pressure 1 bar). The condensed top product (temperature as a rule from 20 to 40° C.) is stabilized with an initiator and comprises substantially desired ester, unconverted alcohol, water, acetate, Michael adduct, such as alkoxypropionate, and various secondary components. The acrylic acid content of the top product is as a rule not more than 0.1%, preferably not more than 0.01%.

The inhibitors used may be alkylphenols, for example o-, m- or p-cresol (methylphenol), 2-tert-butyl-4-methylphenol, 6-tert-butyl-2,4-dimethylphenol, 2,6-di-tert-butyl-4-methylphenol, 2-tert-butylphenol, 4-tert-butylphenol, 2,4-di-tert-butylphenol, 2-methyl-4-tert-butylphenol, 4-tert-butyl-2,6-dimethylphenol or 2,2′-methylenebis(6-tert-butyl-4-methyphenol), hydroxyphenols, for example hydroquinone, 2-methylhydroquinone, 2,5-di-tert-butylhydroquinone, pyrocatechol (1,2-dihydroxybenzene) or benzoquinone, aminophenols, e.g. para-aminophenol, nitrosophenols, e.g. para-nitrosophenol, alkoxyphenols, for example 2-methoxyphenol (guajacol, pyrocatechol monomethyl ether), 2-ethoxyphenol, 2-isopropxyphenol, 4-methoxyphenol (hydroquinone monomethyl ether), mono- or di-tert-butyl-4-methoxyphenol, tocopherols, e.g. α-tocopherol, and 2,3-dihydro-2,2-dimethyl-7-hydroxybenzofuran (2,2-dimethyl-7-hydroxycoumarane), N-oxyls, such as 4-hydroxy-2,2,6,6-tetramethylpiperidin-N-oxyl, 4-oxo-2,2,6,6-tetramethylpiperidin-N-oxyl, 4-acetoxy-2,2,6,-6-tetramethylpiperidin-N-oxyl, 2,2,6,6-tetramethylpiperidin-N-oxyl, 4,4′,4″-tris(2,2,6,6-tetramethylpiperidin-N-oxyl) phosphite or 3-oxo-2,2,5,5-tetramethylpyrrolidin-N-oxyl, aromatic amines or phenylenediamines, e.g. N,N-diphenylamine, N-nitrosodiphenylamine, N,N′-dialkyl-para-phenylenediamine, it being possible for the alkyl radicals to be identical or different and in each case, independently of one another, to consist of 1 to 4 carbon atoms and to be straight-chain or branched, hydroxylamines, e.g. N,N-diethylhydroxylamine, phosphorus-containing compounds, e.g. triphenylphosphine, triphenyl phosphite, hypophosphorous acid or triethyl phosphite, sulfur-containing compounds, e.g. diphenyl sulfide or phenothiazine, if required in combination with metal salts, for example the chlorides, dithiocarbamates, sulfates, salicylates or acetates of copper, manganese, cerium, nickel or chromium.

Water-soluble stabilizers are preferred at this point, particularly preferably N,N′-dialkyl-p-phenylenediamines, e.g. N,N′-diisobutyl-p-phenylenediamine or N,N′-diisopropyl-p-phenylenediamine, N-oxyls, in particular para-phenylenediamine or 4-hydroxy-2,2,6,6-tetramethylpiperidin-N-oxyl.

The stabilizer is used in amounts of 10-1 000 ppm, preferably from 50 to 500 ppm, based on the distillate.

To further support the stabilization, an oxygen-containing gas, preferably air or a mixture of air and nitrogen (air having a low oxygen content), may be present.

This oxygen-containing gas is preferably metered into the bottom region of a column and/or into a circulation evaporator.

Alternatively, it is also possible to dispense with a condensation, in which case the distillate is fed substantially in gaseous form into the downstream scrubbing (stage 3).

The substantially alcohol-free acrylate-containing mixture c1) obtained in stage 3 is applied as reflux to the uppermost column tray. Preferably 20-80, particularly preferably 30 to 60, very particularly preferably 40-60, % by weight, based on the reaction mixture, are applied as reflux.

A part of the esterification bottom product, preferably 0.1-1%, based on desired ester, is separated off continuously as b3) in order to remove high boilers and can either be disposed of, for example incinerated, or fed to a high boiler working-up (stage 8).

The esterification is operated in such a way that the bottom product contains not more than 10% of desired ester and not more than 15% of acrylic acid.

In a further process variant, the esterification is carried out in a heatable preliminary reactor, under atmospheric or superatmospheric pressure, and the liquid reaction mixture thus obtainable is fed to a distillation unit consisting of column, circulation evaporator and condenser. The reaction mixture is separated as described above. The catalyst-containing bottom product is completely or partly recycled into the reactor.

3. Scrubbing

The distillate or condensate obtained in stage 2 and substantially comprising acrylate (75-90%), alcohol (1-10%), water (7-13%), Michael adduct, in particular alkyl alkoxypropionate, (0.5-2.5%), acetate (0.05-1%) and various low boilers, e.g. propionates, aldehydes and ethers, (0.5-3%) is subjected to scrubbing with a wash liquid, if required after addition of further stabilizer.

The amount of wash liquid is 10-200, preferably 40-150, particularly preferably 50-100, % by weight, based on the distillate/condensate.

The wash liquid is, for example, water to which, if required, basic compounds, for example sodium hydroxide, potassium hydroxide, sodium bicarbonate, sodium carbonate, potassium bicarbonate or potassium carbonate, may have been added, water preferably being used.

The wash liquid used may be tap water, condensate or demineralized water, if required provided with the above additives.

A further embodiment comprises using or concomitantly using the aqueous phases obtained in the process, for example from phase separators, for example those from stage 4 or 5, or water from vacuum units, e.g. water ring pumps, in particular the aqueous fraction d2) from the alcohol recovery (stage 4).

What is important according to the invention is that unconverted alcohol from the esterification and other byproducts soluble in the wash liquid are removed by the scrubbing.

In terms of process engineering, in principle all extraction and scrubbing methods and apparatuses known per se can be used for scrubbing in the novel process, for example those which are described in Ullmann's Encyclopedia of Industrial Chemistry, 6th ed, 1999 Electronic Release, Chapter: Liquid—Liquid Extraction—Apparatus. For example, these may be one-stage or multistage, preferably multistage, extractions and those using the cocurrent or countercurrent procedure, preferably the countercurrent procedure.

Columns containing sieve trays, stacked packings or dumped packings, stirred containers or mixer-settler apparatuses and columns having rotating internals or pulsation columns are preferably used.

A column having from 70 to 150 theoretical plates is particularly preferably used. Suitable column internals are in principle all conventional internals, for example trays, stacked packings and/or dumped packings. Among the trays, bubble trays, sieve trays, valve trays, Thormann trays and/or dual-flow trays are preferred; among the dumped packings, those having rings, coils, saddles, Raschig, Intos or Pall rings, barrel or Intalox saddles, Top-Pak, etc. or braids are preferred.

The distillate/condensate from stage 2 is preferably fed in at the lower end of the column and the wash liquid preferably at the top.

The organic phase emerging at the top of the column is passed into a separation vessel known per se, in order to separate off residual water as phase c2), and is stabilized with an inhibitor. The substantially alcohol-free ester phase c1), which as a rule has an alcohol content of not more than 0.1, preferably not more than 0.02, % by weight, is partly fed as reflux to the distillation unit b2) in stage 2 and partly fed to a further distillative purification of the acrylate (stage 5) in the ratio of 30:70-70:30, preferably 40:60-60:40.

According to the invention, the organic phase c1) is worked up by distillation without further scrubbing or neutralizations.

The aqueous phase c2) from the phase separator is preferably fed completely into the alcohol recovery (stage 4).

Here too, the abovementioned alkylphenols, alkoxyphenols, hydroxyphenols, aminophenols, tocopherols, N-oxyls, aromatic amines or phenylenediamines, hydroxylamines, phosphorus-containing compounds or sulfur-containing compounds, if required in combination with metal salts, can be used as inhibitors.

Water-soluble stabilizers, particularly preferably N,N′-dialkyl-para-phenylenediamines and N-oxyls, in particular N,N′-diisobutyl-para-phenylenediamine or 4-hydroxy-2,2,6,6-tetramethylpiperidin-N-oxyl are preferred at this point.

The stabilizer is used in amounts of from 10-1 000 ppm, preferably from 50 to 500 ppm, based on the organic phase emerging at the top of the column.

The aqueous phase c3) emerging at the lower end of the column, which generally contains 5-10% by weight of alcohol, 3-7% by weight of acrylate, 0.1-1% by weight of Michael adduct, such as alkyl alkoxypropionate, and 0.1-1% by weight of low boilers, is completely or partially worked up in stage 4 (alcohol recovery). A part of this aqueous phase c3) can also be recycled as wash liquid into the scrubbing.

A further possibility comprises not condensing the distillate from stage 2 but feeding it in gaseous form to stage 3 and quenching it with the wash liquid.

In order to reduce the amount of wastewater, it may be expedient completely or partly to bypass the scrubbing (stage 3), for example by feeding some or all of the distillate/condensate obtained at the top of the column b2) directly to a distillative working-up, for example to the low boiler removal (stage 5), it being possible, it being possible, if required, for a phase separation vessel to be connected upstream for separating off the water formed in the reaction. The bottom product obtained in the distillative working-up, e.g. e3) can, if required, be partly used as reflux for the column b2).

4. Alcohol Recovery

The aqueous phases c2) and c3) obtained in stage 3 from column and separation container are fed, if required together with the aqueous phases obtained in stage 5 and those obtained in the generation of reduced pressure (water ring pumps), to a recovery of desired product from alcohol, acrylate and Michael adducts.

The recovery unit preferably consists of a distillation column d) with evaporator and condenser, in each case of conventional design, and a side take-off.

The column preferably has 30-70 theoretical plates, for example trays, stacked packings and/or dumped packings. Among the trays, bubble trays, sieve trays, valve trays, Thormann trays and/or dual-flow trays are preferred; among the dumped packings, those having rings, coils, saddles, Raschig, Intos or Pall rings, barrel or Intalox saddles, Top-Pak, etc. or braids are preferred.

The combined aqueous phases are fed in at the upper end of the lower half of the column, the feed being heated to 40-90° C., preferably 60-90° C., preferably by heat exchange with the discharge of the alcohol recovery column.

The bottom temperature is 100-110° C. and the top temperature is 60-80° C. at slightly reduced or atmospheric pressure, preferably at atmospheric pressure.

The vapors emerging at the top of the column are condensed, stabilized with an inhibitor and partly recycled as reflux to the column d). The remaining part of the condensate d1) is fed directly to the esterification (stage 2). If it is liquid, the feed to stage 2 can be metered roughly in the middle of the column b2) into the reactor b1) or into its feed or, if it is gaseous, below the internals having separation activity or into the circulation.

The vapors comprise substantially alcohol (40-70%) and acrylate (30-50%).

Here too, the abovementioned alkylphenols, hydroxyphenols, aminophenols, tocopherols, N-oxyls, aromatic amines or phenylenediamines, hydroxylamines, phosphorus-containing compounds or sulfur-containing compounds, if required in combination with metal salts, can be used as inhibitors.

Water-soluble stabilizers, particularly preferably N,N′-dialkyl-para-phenylenediamines and N-oxyls, in particular N,N′-diisobutyl-para-phenylenediamine or 4-hydroxy-2,2,6,6-tetramethylpiperidin-N-oxyl, are preferred at this point.

The stabilizer is used in amounts of 10-500 ppm, preferably 50-300 ppm, based on the distillate.

To further support the stabilization, an oxygen-cbntaining gas, preferably air or a mixture of air and nitrogen (air having a low oxygen content), may be present.

This oxygen-containing gas is preferably metered into the bottom region of the column and/or into a circulation evaporator.

Only partial condensation is also possible, preferably the part which is required for the reflux, and the gaseous vapors can be passed directly into the esterification.

A medium boiler fraction d3) in gaseous or liquid form, which mainly contains Michael adducts, in particular alkyl alkoxypropionate, (5-10% by weight), alcohol (40-60% by weight) and water, is preferably removed from the column in the lower part of the upper half of the column, via a side take-off, and is fed back to the reactor b1) of the esterification (stage 2). There, the Michael adducts, in particular alkyl alkoxypropionate, are at least partly cleaved back into alcohol and acrylate and the alcohol thus liberated is esterified with the acrylic acid present.

The aqueous phase d2) obtained in the bottom of the recovery column d) is cooled in a heat exchanger, preferably by heat exchange with the column feed of d), at least partly recycled into the scrubbing (stage 3) and used there as wash liquid and partly discharged, preferably in an amount of 1-50%, particularly preferably 5-40%, in particular from 10 to 30%. The discharged part can be disposed of in a manner known per se, for example via a wastewater treatment plant.

A further preferred embodiment comprises applying the fresh alcohol required for the esterification as reflux at the top of the column d) and feeding the vapors directly, preferably in gaseous form, to the reaction zone b) of the esterification (stage 2). The feed is effected as described above.

5. Low Boiler Removal

The low boiler removal is carried out in particular when crude acrylic acid is used as feedstock and/or an acrylate particularly low in low boilers is to be prepared.

A part of the substantially alcohol-free acrylate mixture (organic phase c1) obtained in stage 3 from the phase separator in stage 3 is separated, in a distillation unit e), consisting of a distillation column, an evaporator and a condenser with phase separation vessel, in each case of conventional design, into a low boiler fraction and a bottom product e3) which contains the desired ester.

The column preferably has 20-60 theoretical plates and the internals described in stage 4.

The feed is preferably located above the middle of the column.

The bottom temperature is from 80 to 100° C., and the column is operated at atmospheric or slightly reduced pressure, for example from 500 mbar to atmospheric pressure, preferably from 700 mbar to atmospheric pressure, particularly preferably from 800 mbar to atmospheric pressure.

The condensed low boiler fraction separates into an aqueous phase e2), which is recycled into the alcohol recovery stage (stage 4) and/or into the scrubbing (stage 3), and an organic phase e1), which contains mainly alkyl acetate, ether and alkyl acrylate.

The organic phase e1) is mixed with a stabilizer and is partly applied as reflux to the uppermost column tray of column e) (reflux ratio 20-40) and can partly be fed to a further distillation unit (stage 6).

The abovementioned alkylphenols, alkoxyphenols, hydroxyphenols, aminophenols, tocopherols, N-oxyls, aromatic amines or N,N′-dialkyl-para-phenylenediamines, hydroxylamines, phosphorus-containing compounds or sulfur-containing compounds, if required in combination with metal salts, can be used as inhibitors here.

To further support the stabilization, an oxygen-containing gas, preferably air or a mixture of air and nitrogen (air having a low oxygen content), may be present.

This oxygen-containing gas is preferably metered into the bottom region of the column and/or into a circulation evaporator.

Mixtures of phenothiazine and p-nitrosophenol and/or nitrosodiethylaniline and/or tetramethylpiperidin-1-oxyls and/or N,N′-diisobutyl-para-phenylenediamine are preferably used at this point.

The condenser of stage 5 is preferably fed with a solution of a partially water-soluble inhibitor in the desired ester, preferably by spraying in, in order to prevent polymer formation on the cooling surfaces.

p-Phenylenediamines and N-oxyls, particularly preferably para-phenylenediamine or 4-hydroxy-2,2,6,6-tetramethylpiperidin-N-oxyl, are preferred for this purpose.

6. Low Boiler Working-up

The low boiler working-up is carried out in particular when crude acrylic acid is used as feedstock and/or an acrylate having a particularly low content of low boilers is to be prepared.

That part of the organic phase e1) of the low boiler fraction from stage 5 which is not used as reflux can be separated, in a further distillation unit f) having a condenser and phase separation vessel of conventional design, into a low boiler fraction f1), mainly containing alkyl acrylate and ether, and into a bottom product f2) comprising substantially alkyl acrylate. The condensate f1) is partly removed and, for example, thermally utilized or hydrolyzed and, after stabilization analogously to stage 5, partly recycled as reflux, with a reflux ratio of, for example, 20-40:1, into the distillation column f).

The proportion removed can also be subjected to alkaline hydrolysis, for example with sodium hydroxide solution, in order to recover the alcohol contained in the acetate. The discharge from the hydrolysis can then be fed, for example, to the alcohol recovery d).

The substantially acrylate-containing bottom product f2) of the column is recycled into stage 5.

A further possibility consists in using the bottom product f2) as reflux in the distillation unit b2) of stage 2.

The bottom temperature is from 60 to 80° C. and the pressure is preferably atmospheric or slightly reduced pressure, for example from 500 mbar to atmospheric pressure, preferably from 700 mbar to atmospheric pressure, particularly preferably from 800 mbar to atmospheric pressure.

A preferred embodiment comprises using a packed column having preferably 10-30 theoretical plates.

7. Purifying Distillation

The desired ester is isolated as top product g1) from the bottom product e3) which is obtained in stage 5 and has a purity of, as a rule, at least 98%, preferably at least 99%, or, if stages 5 and 6 were not carried out, from the organic phase c1) in a further distillation stage g) in a distillation column having preferably 5-20 theoretical plates and an evaporator and condenser of conventional design, at a bottom temperature of from 40 to 80° C. and a top pressure of 0.1-0.7, preferably 0.2-0.6, bar. The condenser is fed with a solution of a storage stabilizer in the desired ester, the stabilizer solution preferably being sprayed in.

Suitable column internals for the column are in principle all conventional internals, for example trays, stacked packings and/or dumped packings. Among the trays, bubble trays, sieve trays, valve trays, Thormann trays and/or dual-flow trays are preferred; among the dumped packings, those having rings, coils, saddles, Raschig, Intos or Pall rings, barrel or Intalox saddles, Top-Pak, etc. or braids are preferred.

The abovementioned alkylphenols, alkoxyphenols, hydroxyphenols, aminophenols, tocopherols, N-oxyls, aromatic amines or N,N′-dialkyl-para-phenylenediamines, if required in combination with metal salts, can be used as inhibitors here.

To further support the stabilization, an oxygen-containing gas, preferably air or a mixture of air and nitrogen (air having low oxygen content), may be present.

This oxygen-containing gas is preferably metered into the bottom region of the column and/or into a circulation evaporator.

A preferably used storage stabilizer is hydroquinone monomethyl ether.

The amount is established so that the storage stabilizer content in the condensate is 10-20 ppm.

Some of the desired esters are mixed with the inhibitor mixture from stage 5 (see above), preferably with 5-100 ppm, and are fed as reflux to the column (reflux ratio 0.1-1:1, preferably 0.1-0.7:1, particularly preferably 0.1-0.5:1).

The bottom product of the column g2), which mainly contains alkyl acrylate and Michael adduct, in particular alkyl alkoxypropionate, is preferably fed to the reactor b1), where, under the esterification conditions, the Michael adducts are at least partly cleaved back into alcohol and acrylate.

The acrylates obtainable by the novel process have a purity of at least 99%, preferably at least 99.5%, particularly preferably at least 99.8%, in particular at least 99.9%, and contain not more than 1 000 ppm, preferably not more than 500 ppm, of alkyl propionate, not more than 100 ppm, preferably not more than 50 ppm, of alkyl acetate and not more than 100 ppm, preferably not more than 50 ppm, of acrylic acid.

8. High Boiler Working-up (Optional)

The stream b3) from the esterification (stage 2) can optionally be subjected at least partly to a high boiler cleavage.

For this purpose, the stream b3) or, if required, also the streams d3) and/or g2) containing Michael adduct is or are fed to a reactor or a distillation apparatus operated with, if required, a circulation and is or are treated there thermally and/or catalytically.

The temperature in the cleavage is in general from 110 to 220° C., preferably from 120 to 200° C., particularly preferably from 130 to 180° C.

Separating off the remaining acrylate and the resulting cleavage products can be supported by passing through a gas stream which is substantially inert under the reaction conditions (stripping), for example nitrogen, steam or preferably an oxygen-containing gas, e.g. air.

The residue can, for example, be partly discharged, distilled or subjected again to the cleavage or used for recovering sulfuric acid.

The gas stream which is obtained from the cleavage and may contain substantially acrylate, alcohol, acrylic acid and possibly ether and also stabilizer can, if required, after condensation and/or further cooling, preferably be fed into the esterification reactor b1) or below the internals of b2) which have separation activity or into the circulation of the reaction zone b).

Instead of the high boiler working-up, the stream b3) can also advantageously be used for recovering sulfuric acid.

For this purpose, a sulfur-containing stream is converted in a manner known per se into a stream containing sulfur oxide and is converted into sulfuric acid, for example in a contact plant.

The advantages of the novel process are

possibility of using a crude acrylic acid, i.e. an acrylic acid which may still contain acetic acid, propionic acid, aldehydes and maleic anhydride

only little fouling of the reactor and of the evaporator

reduction of the amounts of wastewater owing to recycling

avoidance of losses of desired product through working-up of the resulting wastewaters and of the low boiler fraction and hence

little environmental pollution

long on-stream times of the distillation units

high yield (at least 98%, based on (meth)acrylic acid)

desired ester having very high purity (>99.9%, aldehyde-free)

no occurrence of solid polymer, e.g. in evaporators

Parts, percentages and ppm data are by weight, unless stated otherwise.

### EXAMPLE 1

Crude acrylic acid having the following composition was used:

99.6% of acrylic acid

0.18% of acetic acid

0.03% of propionic acid

0.05% of furfurals

0.01% of benzaldehyde

0.06% of water

0.01% of maleic anhydride

0.05% of phenothiazine

The crude acrylic acid was treated before the esterification at 23° C. with hydrazine hydrate (1.5 mol/mol of aldehyde) (residence time 1 hour).

5 360 parts of treated crude acrylic acid, 676 parts of bottom product of the purification column, 400 parts of Michael adduct-containing fraction of the methanol recovery and 20 parts of sulfuric acid (96% strength) were fed per hour into the bottom of a column having 40 dual-flow trays and an external circulation evaporator. 4 302 parts per hour of the top product obtained in the methanol recovery were fed in in gaseous form below the 1st tray of the column. 70 parts, mainly containing catalyst, high-boiling byproducts and inhibitors, were discharged continuously from the bottom of the reactor. 7 000 parts of scrubbed crude ester were fed onto the uppermost column tray. The reaction temperature was 130° C.

17 622 parts/h of a mixture which comprised substantially methyl acrylate (82.1%), water (9.5%), methanol (6.5%), low boilers (1.0%) and methyl methoxypropionate (0.9%) were removed at the top of the column and condensed. The condenser was fed with a stabilizer solution (160 parts/h) consisting of a solution of 0.4% of N,N′-diisobutyl-p-phenylenediamine in methyl acrylate.

The crude ester mixture was treated in a scrubber column (120 trays) with 10 000 parts/h of wastewater of the methanol recovery, the aqueous phase of the phase separation vessel of the low boiler distillation (179 parts/h) and the wastewater of the vacuum pumps (580 parts/h) also being added. The scrubbed organic phase was mixed with 160 parts/h of stabilizer solution (see above) and fed partly (7 000 parts/h) as reflux to the reactor column and partly (7 242 parts/h), together with the bottom product of the low boiler working-up (86 parts/h), to the low boiler removal.

The wastewater of the scrubber column was heated to 40° C. and fed onto the 25th tray of the methanol recovery column (60 dual-flow trays, external circulation evaporator, 105° C. bottom temperature). 2 400 parts of methanol (99.9% by weight, fresh feed) as reflux and 100 parts of stabilizer solution (see above) were added at the top of the column. The resulting distillate, which mainly comprises methanol (77.8%) and methyl acrylate (20.6%), was recycled in gaseous form into the esterification. 400 parts/h of a fraction containing Michael adduct were removed in liquid form while a side take-off (40th tray) (6% of methyl methoxypropionate, 48% of methanol and 56% of water) and metered to the esterification reactor.

The methanol-free wastewater was partly recycled into the scrubbing (10 000 parts/h) and partly discharged (2 100 parts/h).

The low boiler removal was effected in a column having 52 dual-flow trays (feed onto the 32nd tray) and a circulation evaporator at a bottom temperature of 88° C. The low boiler fraction removed at the top of the column separated into an aqueous phase (179 parts/h), which was fed to the extraction column, and an organic phase, which was partly recycled as reflux (2 000 parts/h) into the column and partly fed (60 parts/h) to the low boiler working-up. The condenser was fed with 200 parts/h of stabilizer solution consisting of 0.2% of phenothiazine and 0.1% of N,N′-diisobutyl-p-phenylenediamine in methyl acrylate.

The bottom product (7 796 parts/h), mainly comprising methyl acrylate (99%), was separated in the purification column into a top product, the desired ester (7 335 parts/h, 98.5% yield, based on acrylic acid), and a bottom product, which substantially comprised 89% of methyl acrylate and 10.5% of Michael adduct and was recycled to the esterification.

The purifying distillation was effected in a column having 15 dual-flow trays and an external circulation evaporator. The top temperature was 43° C. (0.3 bar) and the reflux ratio was 0.3. 160 parts/h of a solution of 0.2% of hydroquinone monomethyl ether in methyl acrylate was sprayed into the vapor pipe, and the condenser pipe was sprayed with 100 parts/h of this solution. The reflux ratio was 0.3, and 120 parts of stabilizer mixture (see above) were added to the reflux.

The low boiler working-up was effected in a packed column (16 theoretical plates) at a bottom temperature of 72° C. at atmospheric pressure and a reflux ratio of 25. The bottom product, mainly methyl acrylate (94%), was recycled to the low boiler removal, and the top product not used as reflux (19 parts/h) was discharged. 50 parts/h of the stabilizer solution (see above) were added to the reflux of the column.

According to gas chromatographic analysis (analogous to DIN 55 686), the methyl acrylate obtained had a purity of 99.96% and still contained 310 ppm of methyl propionate, 25 ppm of acrylic acid, 60 ppm of water, less than 10 ppm of methyl acetate and less than 1 ppm of aldehydes as secondary components.

The plant could be operated without problems for at least 30 days.

### COMPARATIVE EXAMPLE 1

The procedure was as in example 1, except that the same crude acrylic acid was used without pretreatment with hydrazine.

After 1 week, the plant had to be shut down owing to fouling on the evaporator.

### COMPARATIVE EXAMPLE 2

The procedure was as in example 1, but without the low boiler removal and discharge.

The purity of the product was 99.78% and the product contained 1 700 ppm of methyl acetate and 300 ppm of methyl propionate.

1: A process for the preparation of (meth)acrylates by reacting (meth)acrylic acid having a carbonyl content of less than 50 ppm with an alcohol in the presence of at least one acidic catalyst and at least one polymerization inhibitor in a reaction zone b) consisting of at least one reactor b1) having at least one connected distillation unit b2), wherein
in the reactor b1) the temperature is brought to from 120 to 150° C. and
the desired (meth)acrylic acid: alcohol stoichiometry is established,
the distillate of the distillation unit b2) is treated with a wash liquid (wash step c)),
the discharge from the wash step c) is separated into at least one organic phase c1) and at least two aqueous phases c2) and c3),
some of the organic phase c1) is passed as reflux to the distillation unit b2) and some of it is passed without further washing to a distillative purification g) of the (meth)acrylate and
the aqueous phases c2) and c3) are passed at least partly to an alcohol recovery d).

2: A process as claimed in claim 1, wherein, in the alcohol recovery d), a fraction d1) containing substantially (meth)acrylate and alcohol is separated from the aqueous phases c2) and c3) and said fraction is fed at least partly into the reaction zone b).

3: A process as claimed in claim 1, wherein, in the alcohol recovery d) of the aqueous phases c2) and c3), a substantially water-containing fraction d2) is separated off and said fraction is used at least partly as wash liquid in the wash step c).

4: A process as claimed in claim 1, wherein, in the alcohol recovery d) of the aqueous phases c2) and c3), a fraction d3) containing Michael adducts is separated off and said fraction is fed into the reaction zone b).

5: A process as claimed in claim 1, wherein, before the distillative purification g) of the (meth)acrylate, a low boiler removal e) is carried out, in which a substantially (meth)acrylate-containing fraction e3) is separated from a fraction containing substantially water, alkyl acetate, ether and (meth)acrylate.

6: A process as claimed in claim 5, wherein the fraction containing substantially water, alkyl acetate, ether and (meth)acrylate is separated into an organic phase e1), which substantially contains alkyl acetate, ether and (meth)acrylate, and an aqueous phase e2).

7: A process as claimed in claim 6, wherein the aqueous phase e2) is fed at least partly to the alcohol recovery d) and/or to the wash step c).

8: A process as claimed in claim 6, wherein the organic phase e1) is fed to a low boiler working-up f) and is separated there into a substantially (meth)acrylate-containing fraction f2) and a fraction f1) containing substantially alkyl acetate and ether.

9: A process as claimed in claim 1, wherein the fraction e3) is passed into a purifying distillation g) and is separated there into a fraction g1) containing (meth)acrylate in a purity of at least 99.9% by weight and a fraction g2) containing (meth)acrylate and Michael adduct.

10: A process as claimed in claim 8, wherein the fraction f2) is passed into the lower boiler removal e) and/or is used as reflux into the distillation unit b2).

11: A process as claimed in claim 9, wherein the fraction g2) is passed at least partly into the reaction zone b).

12: A process as claimed in claim 1, wherein the (meth)acrylic acid used is pretreated crude (meth)acrylic acid.

13: A process as claimed in claim 12, wherein the crude (meth)acrylic acid is first treated in a pretreatment a) in the presence of an amine, hydrazine or hydrazine derivative before the esterification.

14: A process as claimed in claim 2, wherein the alcohol fed in is not passed into the reaction zone b) but is used as reflux in the alcohol recovery d) and the fraction d1) is fed into the reaction zone b).

15: A process as claimed in claim 14, wherein the fraction d1) is fed in gaseous form into the reaction zone b).

16: A process as claimed in claim 1, wherein the alcohol used is methanol or ethanol and the (meth)acrylic acid used is acrylic acid.
